(19) United States
(12) Patent Application Publication
(10) Pub. No.: US 2026/0102791 A1
(43) Pub. Date: Apr. 16, 2026

(54) SPRAY DEVICE

(71) Applicant: Medtronic, Inc., Minneapolis, MN (US)

(72) Inventor: David T. Passione, Richboro, PA (US)

(21) Appl. No.: 19/356,672

(22) Filed: Oct. 13, 2025

Related U.S. Application Data
(60) Provisional application No. 63/707,021, filed on Oct. 14, 2024.

(51) Int. Cl. B08B 3/02; A61B 90/70; G02B 25/00

(52) U.S. Cl. CPC B08B 3/02; A61B 90/70; B08B 2203/0211; G02B 25/002

(57) ABSTRACT

A medical system including an apparatus configured to deliver one or more fluid sprays to a portion of a medical device, such as an attachment member of the medical device. The apparatus defines an inner chamber in which the medical device may be positioned to receive the one or more fluid sprays. The apparatus includes a fitting configured to receive a fluid comprising the fluid sprays from a fluid delivery device. The apparatus further includes a magnification pane configured to provide visual access to the medical device when the medical device is positioned within the inner chamber. In examples, the magnification pane is configured to provide a magnified apparent image of the medical device within the inner chamber.

### RELATED APPLICATIONS

This application claims the benefit of U.S. Provisional Application Serial Number 63/707,021 filed Oct. 14, 2024, the entire contents of each of which are incorporated herein by reference.

### TECHNICAL FIELD

This disclosure is related to an apparatus for the application of a fluid spray to a medical device.

### BACKGROUND

Implantable medical devices are often placed in a subcutaneous pocket and coupled to one or more transvenous medical electrical leads carrying pacing and sensing electrodes positioned in the heart. In some examples, an intracardiac pacemaker may be implantable within a ventricular chamber of a patient’s heart for delivering ventricular pacing pulses without the use of electrical leads. Such pacemakers or other implantable medical devices may also be able to detect the occurrence of arrhythmias, such as fibrillation, tachycardia and bradycardia, in the patient’s heart. An implantable cardiac defibrillator may deliver electrical shocks to the patient’s heart in response to detection of a tachycardia or fibrillation to restore a normal heartbeat in the patient. In some cases, a single implantable medical device functions as both an implantable pacemaker and implantable cardiac defibrillator.

Implantable medical devices may include electrodes and/or other elements for physiological sensing and/or therapy delivery. The electrodes and/or other elements may be implanted at target locations selected to detect a physiological condition of the patient and/or deliver one or more therapies. For example, the electrodes and/or other elements may be delivered to a target location within an atrium or ventricle to sense intrinsic cardiac signals and deliver pacing or antitachyarrhythmia shock therapy from a medical device coupled to a lead.

### SUMMARY

This disclosure describes an apparatus configured to impact a portion of a medical device (e.g., an attachment member) with one or more sprays of a fluid to, for example, remove tissue and/or other anatomical matter from the medical device. The apparatus defines an inner chamber in which the medical device may be positioned (e.g., via an entry channel) to receive the one or more fluid sprays. The apparatus includes a fitting configured to receive a fluid comprising the fluid sprays from a fluid delivery device, such as a syringe-type type. The apparatus further includes a magnification pane configured to provide visual access to the medical device when the medical device is positioned within the inner chamber, such that a clinician may, for example, evaluate the removal of the matter from the medical device. In examples, the magnification pane is configured to provide a magnified apparent image of the medical device within the inner chamber.

In examples, an apparatus comprises: a housing, the housing defining an outer surface and an inner surface opposite the outer surface, the inner surface defining an inner chamber, and the housing defining a longitudinal axis extending through the inner chamber, wherein: the housing defines an entry passage opening into the inner chamber and intersected by the longitudinal axis, the entry passage extending from the outer surface to the inner surface, the housing supports one or more fluid channels, each fluid channel fluidly coupled to a spray outlet which opens into the inner chamber, and each spray outlet configured to provide a spray of a fluid into the inner chamber when the one or more fluid channels receives the fluid, and the housing supports a magnification pane configured to provide visual access through the housing and into the inner chamber; and a fitting defining a fluid chamber configured to hold the fluid, wherein the fluid chamber is configured to provide the fluid to the one or more fluid channels.

In examples, an apparatus comprises: a housing, the housing defining an outer surface and an inner surface opposite the outer surface, the inner surface defining an inner chamber, and the housing defining a longitudinal axis extending through the inner chamber, wherein: the housing defines an entry passage intersected by the longitudinal axis, the entry passage extending from the outer surface to the inner surface, the housing supports a one or more fluid channels, each fluid channel defining a spray outlet which opens into the inner chamber, and each spray outlet configured to provide a spray of a fluid into the inner chamber when the one or more fluid channels receives the fluid, the housing supports a magnification pane configured to provide visual access through the housing and into the inner chamber, the housing supports the magnification pane to cause a field of view of the magnification pane to encompass a portion of the inner chamber through which the longitudinal axis extends, and the magnification pane defines an optical axis substantially perpendicular to the longitudinal axis; and a fitting defining a fluid chamber configured to hold the fluid, wherein the fluid chamber is configured to provide the fluid to the one or more fluid channels.

In examples, a technique comprises: providing, using a fitting defining a fluid chamber, a fluid to a one or more fluid channels supported by a housing; providing, using the one or more fluid channels, at least one spray of a fluid into an inner chamber defined by an inner surface of the housing, wherein the housing defines a longitudinal axis extending through the inner chamber, wherein the housing defines an entry passage opening into the inner chamber and intersected by the longitudinal axis, and wherein the entry passage extends between the inner surface and an outer surface defined by the housing and opposite the inner surface; and providing, using a magnification pane supported by the housing, visual access through the housing and into the inner chamber.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example apparatus configured to provide one or more fluid sprays to a medical device.

FIG. 2 is a perspective view of the apparatus in accordance with the X-Y-Z axes shown.

FIG. 3 is a schematic plan view of the apparatus in accordance with the X-Y-Z axes shown.

FIG. 4 is a schematic end view of the apparatus of FIG. 2 in accordance with the X-Y-Z axes shown.

FIG. 5 is a cross-sectional plan view of the apparatus of FIG. 3 and FIG. 4, the cross-section taken with a cutting plane parallel to a X-Y plane defined by the X-Y-Z axes shown.

FIG. 6 is a cross-sectional end view of the apparatus of FIG. 3, FIG. 4, and FIG. 5, the cross-section taken with a cutting plane parallel to a Y-Z plane defined by the X-Y-Z axes shown.

FIG. 7 is a perspective view of an example of the apparatus in accordance with the X-Y-Z axes shown.

FIG. 8 is a schematic plan view of a magnification pane in accordance with the X-Y-Z axes shown.

FIG. 9 is a schematic end view of the magnification pane of FIG. 8 in accordance with the X-Y-Z axes shown.

FIG. 10 is a schematic view of a portion of a first example fitting.

FIG. 11 is a schematic view of a portion of a second example fitting.

FIG. 12 illustrates an example technique for using the apparatus.

### DETAILED DESCRIPTION

This disclosure describes an apparatus configured to provide one or more sprays of a fluid to an attachment member of a medical system. The apparatus may provide the spray of fluid to the attachment member to, for example, substantially clean the attachment member following or prior to an attempted implantation of the medical system. For example, during a procedure, a clinician may cause the attachment member of the medical system to contact and/or implant within tissue at several sites within an anatomical volume (e.g., a heart chamber) of a patient in order to confirm a sufficient location for the implantation (e.g., based on signals received by processing circuitry when the attachment member contacts tissue at a given location). As the attachment device is implanted and subsequently removed from one or more sites, tissues and/or other anatomical matter may collect on the attachment device, potentially impacting an effectiveness and/or precision with which the attachment device fixates.

The apparatus disclosed is configured to provide the spray of fluid to remove the tissues and/or other anatomical matter at the clinician may deem necessary. For example, the clinician may withdraw a medical device housing (e.g., a medical lead housing) and the attachment member supported thereby from the anatomical volume to a location extracorporeal to the patient. The clinician may use the apparatus to substantially remove tissues and/or other anatomical matter from the attachment member before, for example, re-introducing the medical device housing and the attachment member to the anatomical volume to affix the attachment to a subsequent location.

The apparatus may be configured to position within a boundary of a sterile field (e.g., a sterile space) established and maintained during an implantation procedure, such that the clinician may use the apparatus to clean the attachment within the sterile field. In examples, the apparatus includes a housing (“apparatus housing”) configured to assist in maintaining the apparatus within the sterile field (e.g., using a weight of the apparatus housing and/or one or more feet of the apparatus housing). The apparatus may be configured such that the clinician may insert the attachment member (and/or some portion of the medical device housing supporting the attachment member) within an inner chamber defined by the apparatus housing. The apparatus is configured to provide the spray of fluid as the attachment member is within the inner chamber to substantially remove tissues and/or other anatomical matter from the attachment member. In examples, the apparatus housing defines a fitting configured to receive the fluid from a device configured to deliver the fluid (e.g., a syringe-type device). Hence, the apparatus may be configured such that a removal of tissues and/or other anatomical matter from the attachment member may be conducted using an apparatus housing and a fluid delivery device both configured to remain within the boundary of a sterile field during the implantation procedure.

The apparatus housing supports a magnification pane configured to allow the clinician to visually observe the attachment member within the inner chamber. The magnification pane is configured to enlarge the apparent size of the attachment member within the inner chamber, such that clinician may evaluate the removal of tissues and/or other anatomical matter. The apparatus is configured such that a field-of-view (FOV) defined by the magnification pane includes a portion of the inner chamber into which the attachment member is extended in preparation for or during the removal of tissues and/or other anatomical matter by the spray of fluid. In some examples, the apparatus includes a visual marker (e.g., a marker on a surface of the magnification pane) configured to appear within the FOV of the magnification pane. The visual marker may be configured to demarcate a boundary (e.g., a geometric plane) within the inner chamber to, for example, serve as an observable limit for the extension of a medical device (e.g., a medical lead) into the inner chamber. For example, the visual marker may demarcate the boundary such that the attachment member positions within the inner chamber substantially at a location where the fluid spray is expected to more efficiently remove the tissues and/or other anatomical matter. The visual marker may demarcate the boundary to limit and/or avoid contact between the attachment member (e.g., a distal end of the attachment member) and an inner surface of the housing defining the inner chamber to, for example, avoid deformation and/or damage to the attachment member.

The apparatus housing supports one or more fluid channels configured to deliver the fluid from the fitting (e.g., a fluid chamber defined by the fitting) to the inner chamber to cause the one or more sprays of the fluid within the inner chamber. In examples, each fluid channel defines a spray outlet configured to provide a fluid spray to the inner chamber to cause the one or more sprays of the fluid within the inner chamber. For example, the apparatus housing may define an inner surface defining the inner chamber. A spray outlet may substantially open to or extend substantially through the inner surface to allow the spray outlet to provide the fluid spray to the inner chamber. In examples, the one or more spray outlet are configured to direct the fluid spray toward an attachment member when the attachment member is positioned within the FOV of the magnification pane.

In examples, the apparatus housing defines a longitudinal axis extending through the inner chamber. The apparatus may define an entry passage configured to receive at least some portion of the medical device housing and the attachment member supported thereby. In examples, the longitudinal axis extends through the entry passage. The apparatus may be configured such that the attachment member (and, e.g., the portion of the medical device housing) may be transited through the entry passage (e.g., by a clinician) and into the inner chamber in a direction substantially along the longitudinal axis. A spray outlet may be configured to direct its fluid spray toward the longitudinal axis to cause its fluid spray to impact the attachment member.

In examples, the longitudinal axis extends through the FOV of the magnification pane. In examples, the visual marker of the apparatus is configured to substantially demarcate a position on the longitudinal axis. Hence, in examples, the apparatus is configured such that the attachment member (and, e.g., the portion of the medical device housing) may be extended into the inner chamber to a position proximate the position on the longitudinal axis, and such that the attachment member positions at a location where the fluid spray may more efficiently remove the tissues and/or other anatomical matter, and/or where contact between the attachment member and the inner surface defining the inner chamber is mitigated and/or avoided.

In examples, the apparatus supports a plurality of fluid outlets each configured to direct a fluid spray toward the longitudinal axis. In examples, the plurality of fluid outlets are arranged around a perimeter surrounding the longitudinal axis, such that the plurality of fluid outlets direct fluid sprays toward the longitudinal axis along a plurality of radial directions. Hence, the plurality of fluid outlets may be configured to provide fluid sprays which impact the attachment member around a circumference of the attachment member to assist in the removal of tissues and/or other anatomical matter. In examples (e.g., in addition to the arrangement around the perimeter), the plurality of fluid outlets may be distributed along an axial direction substantially parallel to the longitudinal axis, such that the plurality of fluid outlets direct fluid sprays toward the longitudinal axis over the axial direction. Hence, the plurality of fluid outlets may be configured to provide fluid sprays which impact the attachment member over an axial length of the attachment member to assist in the removal of tissues and/or other anatomical matter.

In some examples, the inner surface of the apparatus housing includes an end surface substantially facing the entry passage. The end surface may be configured to mitigate and/or avoid deformation and/or damage to the attachment member should the attachment member contact the end surface. In examples, the end surface is configured to deform (e.g., to elastically deform) if distal end of the attachment member contacts the end surface and imparts a force to the end surface (e.g., a force transferred to the attachment member by the medical device housing during extension of the attachment member into the inner chamber). In some examples, the end surface is configured to assist in positioning the attachment member within the inner chamber (e.g., instead of or in addition to the visual marker of the apparatus). For example, the end surface may be configured such that the attachment member positions at a location where the fluid spray is expected to more efficiently remove the tissues and/or other anatomical matter when the attachment member contacts the end surface. The end surface may be configured to substantially “give” when the attachment member contacts the end surface to, for example, mitigate and/or avoid deformation and/or damage to the attachment member.

In examples, the apparatus is configured to provide the spray of fluid to an attachment member configured to implant within an anatomical volume (e.g., a heart chamber) of a patient for the delivery of therapy to the patient and/or the sensing of signals from the patient. In examples, the attachment member is supported by a medical device housing and extends distal to a distal end of the medical device housing. In examples, the medical device housing is a housing of a medical lead. For example, the medical lead may be configured such that a distal portion of the medical lead is implanted within the anatomical volume as a proximal portion of the medical lead communicates with processing circuitry supported by a device of the medical system positioned outside the anatomical volume (e.g., a subcutaneous implanted pacemaker). In some examples, the medical device housing mechanically supports processing circuitry configured to reside with the anatomical volume when the attachment member implants within the anatomical volume (e.g., an intracardiac pacemaker). The medical device housing may be configured to position the attachment member at least partially within a heart of a patient, such as within an atrium, ventricle, coronary sinus, or other portions of the heart. In examples, the attachment member is configured to implant within and/or establish contact between at least one electrode and tissue of the patient, such as a septal wall of the heart. In examples, the attachment device is configured to implant within the tissue when the medical device housing is rotated (e.g., by a clinician) about an axis defined by the medical device housing( e.g., the attachment member may be a helical body).

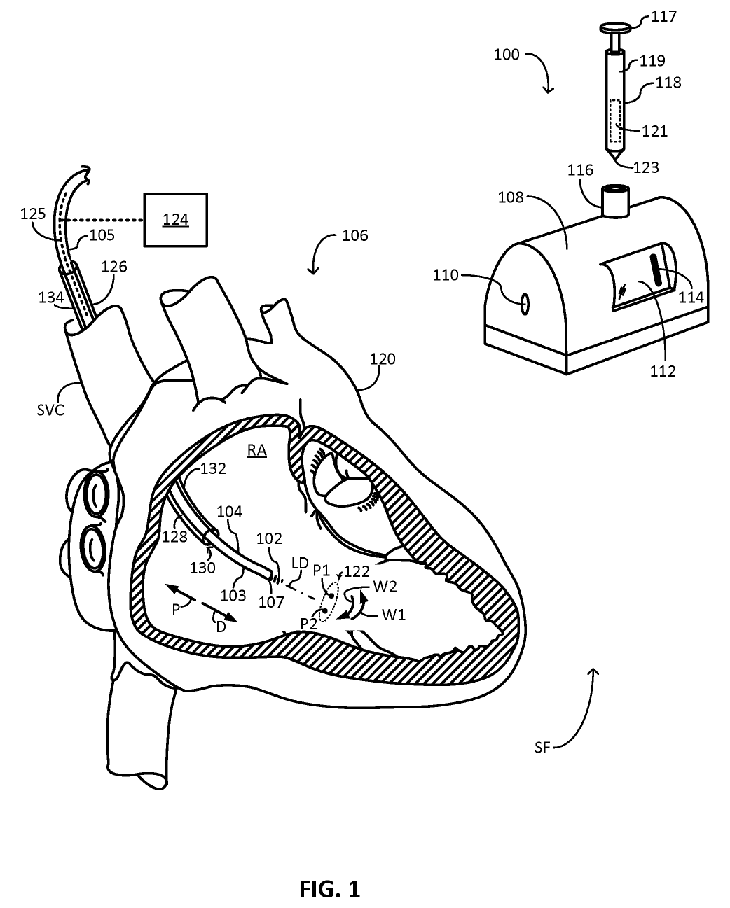

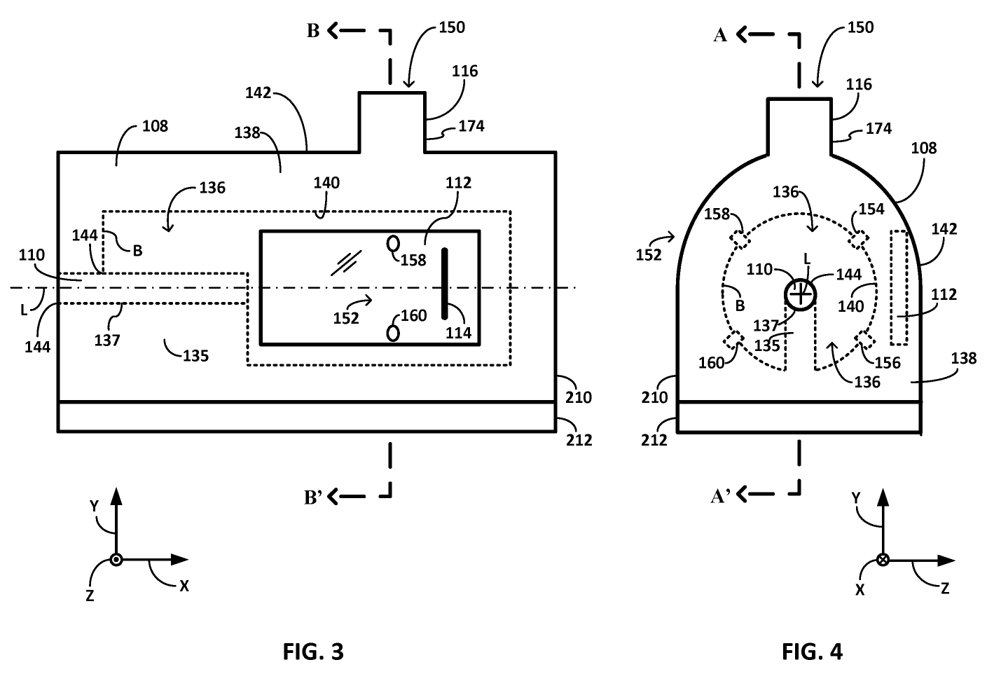

For example, FIG. 1 is a conceptual diagram illustrating an example apparatus 100 configured to deliver a spray of a fluid to an attachment member 102 supported by a medical device housing 104 of a medical system 106. Apparatus 100 includes a housing 108 (“apparatus housing 108”) defining an inner chamber (e.g., inner chamber 136 (FIGS. 3 - 6)). Apparatus 100 is configured to provide one or more fluid sprays which impact attachment member 102 when attachment member 102 is positioned within the inner chamber. Apparatus housing 108 defines an entry passage 110 configured to receive attachment member 102 (and, e.g., some portion of medical device housing 104) and allow the extension of attachment member 102 and/or the portion of medical device housing 104 into the inner chamber.

Apparatus 100 is configured to deliver the spray of fluid toward attachment member 102 to, for example, substantially remove tissues and/or other anatomical matter from attachment member 102. Apparatus housing 108 supports a magnification pane 112 configured to allow observation of attachment member 102 within the inner chamber when entry passage 110 receives attachment member 102 (and, e.g., some portion of medical device housing 104). Magnification pane 112 is configured to enlarge the apparent size of attachment member 102. Magnification pane 112 defines a field-of-view (FOV) which encompasses a portion of the inner chamber into which attachment member 102 is extended. In examples, apparatus 100 includes a visual marker 114 configured to appear within the FOV of magnification pane 112. Visual marker may be configured to demarcate a boundary (e.g., a geometric plane) within the inner chamber to, for example, serve as an observable limit for the extension of attachment member 102 into the inner chamber. In some examples, magnification pane 112 supports and/or otherwise defines visual marker 114 (e.g., on a surface or within a body of magnification pane 112), although this is not required.

In examples, apparatus 100 includes a fitting 116 configured to receive the fluid from a fluid delivery device 118 configured to deliver the fluid. In some examples, fluid delivery device 118 is a syringe-type device. Hence, apparatus 100 may be configured such that a clinician may insert attachment member 102 and a portion of medical device housing 104 into the inner chamber to, for example, remove tissue and/or other anatomical matter from attachment member 102. In examples, the clinician may position attachment member 102 within the inner chamber using visual marker 114. The clinician may use fluid delivery device 118 to provide a fluid (e.g., a pressurized fluid) to apparatus 100 using fitting 116, such that apparatus 100 directs one or more sprays of fluid toward attachment member 102 using the fluid. The clinician may observe the enlarged apparent size of attachment member 102 using magnification pane 112 to evaluate the removal of tissue or other anatomical matter from attachment member 102 during and/or subsequent to apparatus 100 directing the one or more sprays of fluid toward attachment member 102.

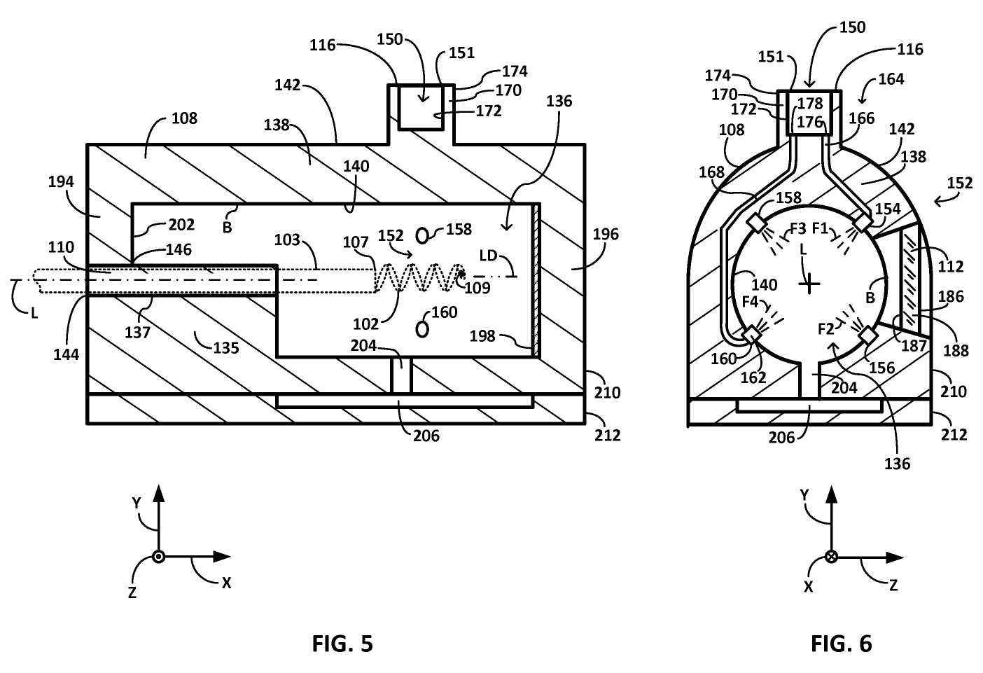

Attachment member 102 is configured to implant within tissues of an anatomical volume defined by a patient. For example, medical system 106 may be configured to position medical device housing 104 and attachment member 102 within an anatomical volume such as a right atrium (“RA”) of a heart 120. Medical system 106 may be configured to deliver and/or retrieve attachment member 102 to and/or from the vicinity of a target site 122 of heart 120. In examples, medical system 106 is configured such that a clinician may cause attachment member 102 to contact and/or implant within tissue in proximity to target site 122 in order to confirm a sufficient location for an implantation. For example, medical system 106 (e.g., attachment member 102) may be configured to communicate with processing circuitry 124 to sense signals generated by a patient (e.g., generated by heart 120) at least when attachment member 102 is in contact with tissues within target site 122. In examples, attachment member 102 and/or medical device housing 104 supports one or more electrodes (e.g., electrode 109 (FIG. 5)) configured to sense signals generated by the patient. In examples, medical system 106 includes a conductor 125 configured to electrically connect the one or more electrodes and processing circuitry 124.

Medical system 106 may be configured such that a clinician may evaluate the placement of attachment member 102 subsequent to contact between attachment member 102 and tissues within target site 122. For example, the clinician may evaluate the placement of attachment member 102 (e.g., using processing circuitry 124) at first tissue location P1 within target site 122. Medical system 106 may be configured such that the clinician may cause the withdrawal of attachment member 102 from first tissue location P1 and move attachment member 102 (e.g., using medical device housing 104) to a second tissue location P2 within target site 122, for example, to evaluate the placement of attachment member 102 at second tissue location P2 (e.g., using processing circuitry 124). Medical system 106 is configured such that attachment member 102 (and medical device housing 104) may be withdrawn from heart 120 and positioned within apparatus 100 for the removal of tissue and/or anatomical matter from attachment member 102 (if the clinician desires).

For example, following the withdrawal of attachment member 102 from first tissue location P1, a clinician might chose to withdraw attachment member 102 and medical device housing 104 from its intracorporeal position within the patient (e.g., through vasculature of the patent) to remove any tissue and/or anatomical matter that might be present on attachment member 102 (e.g., prior to implanting attachment member 102 at second tissue location P2). The clinician may use apparatus 100 (e.g., positioned within a sterile field SF) to deliver one or more fluid sprays to attachment member 102 for the removal of the tissue and/or anatomical matter. The clinician may position attachment member 102 within the inner chamber of apparatus 100 using entry passage 110 and, for example, visual marker 114. In examples, the clinician may use fluid delivery device 118 (e.g., a syringe-type device within sterile field SF) to provide a fluid to apparatus 100 and cause the fluid sprays. Following delivery of the fluid sprays by apparatus 100, the clinician may evaluate the removal of tissue and/or other anatomical matter using magnification pane 112 before, for example, re-introducing attachment member 102 into the patient for implantation at second tissue location P2.

Fluid delivery device 118 is configured to pressurize a fluid held by fluid delivery device 118 when fluid delivery device 118 delivers the fluid to fitting 116. In examples, fluid delivery device 118 is configured such that a clinician may cause the pressurization of the fluid within fluid delivery device 118. For example, in some examples, fluid delivery device 118 is a syringe-type device including a plunger 117 and a barrel 119. Barrel 119 may define a device chamber 121 and a device opening 123 which opens in the device chamber 121. Fluid delivery device 118 may be configured such that when plunger 117 moves (e.g., is moved by a clinician) relative to barrel 119, plunger 117 pressurizes a fluid within device chamber 121 and drives the fluid through device opening 123. Apparatus 100 may be configured to fluidically couple a fluid chamber defined by fitting 116 (e.g., fluid chamber 150 (FIGS. 3-6) and device chamber 121 (e.g., via device opening 123) when fitting 116 receives barrel 119. Hence, Apparatus 100 may be configured such that a clinician may insert fluid delivery device 118 into fitting 116 and move plunger 117 relative to barrel 119 to cause delivery of a pressurized fluid via device opening 123 to fitting 116. Fitting 116 may be configured to deliver the fluid sprays to the inner chamber of apparatus 100. Fluid delivery device 118 may be configured to deliver the fluid to fitting 116 in other manners in other examples.

Medical device housing 104 may include a distal portion 103 (“medical device distal portion 103”) and a proximal portion 105 (“medical device proximal portion 105”). Medical device distal portion 103 may support attachment member 102. In examples, attachment member 102 is coupled (e.g., mechanically coupled) to medical device distal portion 103. In some examples, medical device distal portion 103 defines a distal end 107 (“medical device distal end 107”). Attachment member 102 may be configured to (e.g., supported by medical device housing 104 to) extend distal to medical device distal end 107 (e.g., extend in the distal direction D).

In some examples, for example when attachment member 102 and medical device housing 104 comprise a medical lead, medical device housing 104 is configured such that medical device distal portion 103 may be intracorporeal to the patient and medical device proximal portion 105 may be extracorporeal to the patient when medical device distal portion 103 is intracorporeal. In some examples, for example when attachment member 102 and medical device housing 104 comprise a medical device configured to position processing circuitry 124 within the anatomical volume (e.g., an intracardiac pacemaker), medical device housing 104 may be configured such that medical device proximal portion 105 may be intracorporeal to the patient (e.g., within the anatomical volume) when medical device distal portion 103 is intracorporeal to the patient (e.g., within the anatomical volume).

In examples, medical system 106 includes a delivery catheter 126 configured to deliver and/or retrieve attachment member 102 and/or medical device housing 104 to and/or from the vicinity of target site 122 of heart 120. In FIG. 1, delivery catheter 126 is depicted as transparent for clarity. Delivery catheter 126 may define a lumen 128 (“delivery catheter lumen 128”) configured to deliver attachment member 102 and/or medical device housing 104 to the anatomical volume of the patient (e.g., the RA). In examples, attachment member 102 and/or medical device housing 104 are configured to slidably translate within delivery catheter lumen 128. In examples, delivery catheter 126 defines an opening 130 (“delivery catheter opening 130) which opens into delivery catheter lumen 128. Delivery catheter opening 130 may be configured to allow at least attachment member 102 and/or medical device housing 104 to pass therethrough. In examples, delivery catheter 126 may include a distal portion 132 (“delivery catheter distal portion 132”) configured to be intracorporeal to the patient and a proximal portion 134 (“delivery catheter proximal portion 134”) which may be extracorporeal to the patient when delivery catheter distal portion 132 is intracorporeal.

In some examples, for example when attachment member 102 and/or medical device housing 104 comprise a medical device configured to position processing circuitry 124 within the anatomical volume (e.g., an intracardiac pacemaker), delivery catheter 126 may support a device receptacle (not shown) defining a receptacle volume configured to hold the medical device (e.g., during delivery to and/or retrieval from the anatomical volume). Delivery catheter 126 may be configured such that the receptacle volume comprises some portion of delivery catheter lumen 128. In examples, the receptacle device defines delivery catheter opening 130.

In examples, medical system 106 is configured to deploy at least attachment member 102 and/or medical device housing 104 through delivery catheter opening 130 to cause attachment member 102 to engage tissues within target site 122. In examples, medical system 106 is configured to cause attachment member 102 to disengage from tissues within target site 122 to, for example, retrieve attachment member 102 and/or medical device housing 104 from heart 120 and/or reposition attachment member 102 and/or medical device housing 104 within heart 120. In some examples, In examples, delivery catheter 126 is configured to deliver and/or retrieve attachment member 102 and medical device housing 104 using vasculature, such as an SVC or other vasculature leading to the anatomical volume. In examples, medical device housing 104 and attachment member 102 are configured to transit through the vasculature (e.g., be transited using delivery catheter 126).

Attachment member 102 may be configured to secure medical device housing 104 to tissues of heart 120. Attachment member 102 is configured to penetrate tissue of heart 120 at or near a target site, such as a target site 122. For example, attachment member 102 may be configured to penetrate cardiac tissue of a septal wall in a RV, RA, LV, and/or LA of heart 120, or penetrate cardiac tissue in another area of heart 120. Attachment member 102 may be configured to substantially maintain medical device housing 104 at or in the vicinity of target site 122 when attachment member 102 penetrates tissues at or in the vicinity of target site 122. In some examples, attachment member 102 defines a helical body configured to implant within a tissue wall (e.g., within target site 122) when medical device housing 104 causes attachment member to rotate in one of a first rotational direction W1 or a second rotational direction W2 about a device axis LD defined by medical device housing 104. The helical body may be configured to withdraw from the tissue wall when medical device housing 104 causes attachment member to rotate in the other of first rotational direction W1 or second rotational direction W2 about device axis LD. In other examples, attachment device 104 may define another shape, such as an elongate tine supported by medical device distal portion 103 and configured to extend distal to medical device distal end 107, a thread spiraling around medical device distal portion 103, or some other shape.

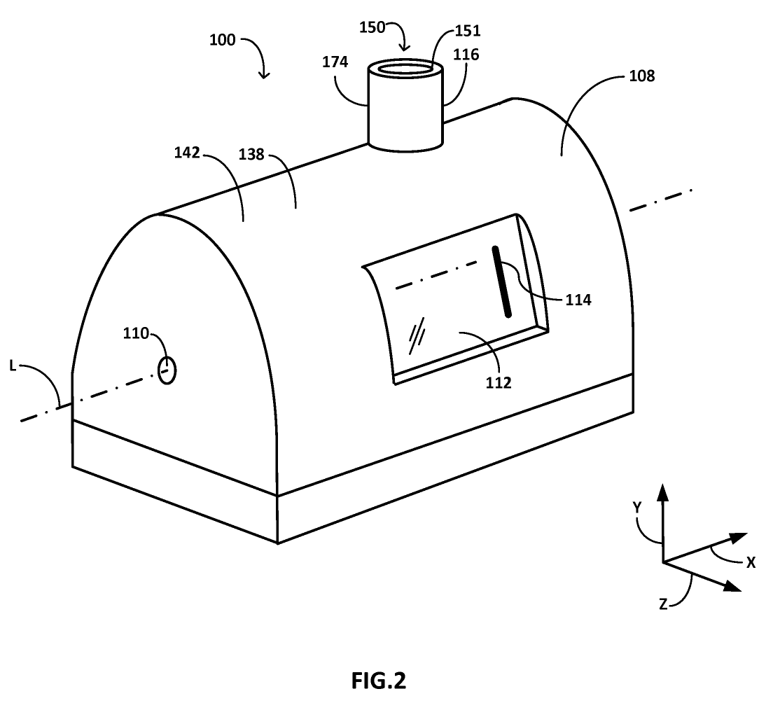

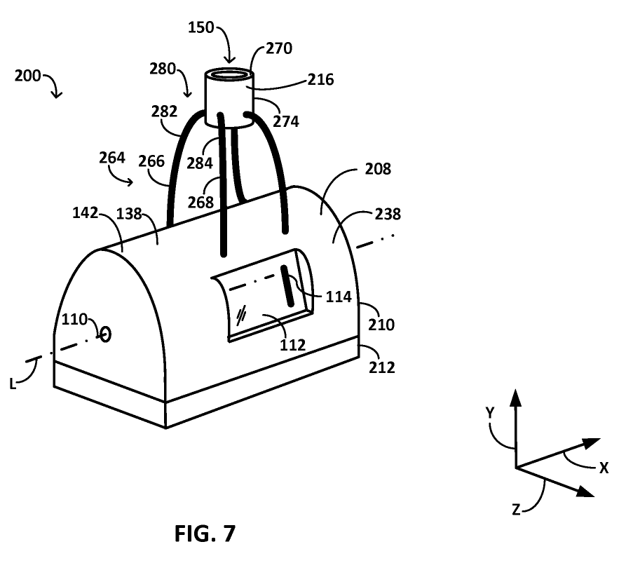

FIG. 2 is a perspective view of apparatus 100, illustrated in accordance with the X-Y-Z axis shown. FIG. 3 is a schematic plan view of apparatus 100 illustrated in accordance with the X-Y-Z axis shown, with the Z axis proceeding out of the page. FIG. 4 is a schematic end view of apparatus 100 illustrated in accordance with the X-Y-Z axis shown, with the X axis proceeding into the page. FIG. 3 and FIG. 4 depict selected hidden lines as dashed lines. FIG. 5 is a schematic cross-sectional view of apparatus 100 taken with a cutting plane A-A' of FIG. 4 and illustrated in accordance with the X-Y-Z axis shown, with the Z axis proceeding out of the page. FIG. 5 also depicts medical device housing 104 and attachment member 102 with dashed lines and extended into apparatus 100, for reference. FIG. 6 is a schematic cross-sectional view of apparatus 100 taken with a cutting plane B-B' of FIG. 3 and illustrated in accordance with the X-Y-Z axis shown, with the X axis proceeding into the page. FIG. 7 is a perspective view of an apparatus 200, which is an example of apparatus 100, and which is illustrated in accordance with the X-Y-Z axis shown.

Apparatus housing 108 defines an inner chamber 136 (e.g., a volume) configured to receive at least a portion of a medical device, such as medical device distal portion 103 and/or attachment member 102. In examples, apparatus housing 108 includes a wall 138 defining an inner surface 140 and an outer surface 142 opposite inner surface 140. In examples, inner surface 140 defines at least a portion of a boundary B surrounding inner chamber 136. Apparatus 100 defines entry passage 110 extending through a portion of apparatus housing 108 and opening into inner chamber 136. In examples, entry passage 110 extends from an inlet 144 (“entry inlet 144”) defined by apparatus housing 108 to an outlet 146 (“entry outlet 146”) defined by apparatus housing 108. In examples, entry inlet 144 is defined and/or supported by a portion of outer surface 142. Entry outlet 146 may be defined and/or supported by a portion of inner surface 140. In some examples, inner surface 140 may support and/or define visual marker 114. For example, inner surface 140 may support and/or define visual marker 114 on a portion of inner surface visible within the field-of-view of magnification pane 112, such that visual marker 114 is visible within the field-of-view of magnification pane 112.

Apparatus housing 108 defines a longitudinal axis L extending through inner chamber 136. In examples, longitudinal axis L extends through entry passage 110 (e.g., extends through entry inlet 144 and entry outlet 146). Apparatus housing 108 is configured to receive at least some portion of a medical device via entry passage 110 such that the portion of the medical device positions within inner chamber 136. For example (as depicted in FIG. 5), apparatus housing 108 may be configured to receive a medical device comprising medical device distal portion 103 and attachment member 102 via entry passage 110 such that medical device distal portion 103 and attachment member 102 position with inner chamber 136.

Apparatus 100 is configured to deliver one or more fluid sprays into inner chamber 136 to, for example, remove matter (e.g., substantially clean) a medical device inserted into inner chamber 136. For example (e.g., as depicted in FIG. 6), apparatus 100 may be configured to deliver one or more of fluid spray F1, fluid spray F2, fluid spray F3, and/or fluid spray F4 into inner chamber 136. In examples, apparatus 100 is configured to receive a fluid using fitting 116 and deliver fluid sprays F1, F2, F3, Fb using the fluid. Fitting 116 may define a fluid chamber 150 configured to receive the fluid (e.g., from fluid delivery device 118 (FIG. 1)). Hence, apparatus 100 may be configured to deliver fluid spray F1, F2, F3, Fb to inner chamber 136 (e.g., to remove matter from attachment member 102) when attachment member 102 is inserted within inner chamber 136 via entry passage 110 and fluid chamber 150 receives a fluid (e.g., from fluid delivery device 118).

In some examples, apparatus 100 (e.g., apparatus housing 108) defines and/or supports a guide portion 135 configured to assist in an insertion of a medical device (e.g., medical device distal portion 103 and/or attachment member 102) into inner chamber 136 via entry passage 110. Guide portion 135 may be configured to assist in positioning the medical device (e.g., attachment member 102) within inner chamber 136 at a location where an effectiveness of fluid spray F1, F2, F3, F4 for the removal of matter might be enhanced (e.g., a location where impact of fluid spray F1, F2, F3, F4 on the medical device might be enhanced). In examples, guide portion 135 defines a guide surface 137 configured to support the medical device (e.g., medical device distal portion 103 and/or attachment member 102) as the medical device is inserted via entry passage 110. For example, guide surface 137 may be configured such that at least medical device distal portion 103 slidably translates over guide surface 137 as medical device distal portion 103 and attachment member 102 are inserted into inner chamber 136 via entry passage 110. Guide surface 137 may be configured to support at least medical device distal portion 103 such that device axis LD remains substantially parallel to and/or coincident with longitudinal axis L during the slidable translation. In some examples, at least some portion of guide surface 137 is substantially parallel to and/or coincident with longitudinal axis L.

In examples, apparatus housing 108 includes one or more spray outlets 152 (“spray outlets 152) configured to deliver fluid spray F1, F2, F3, F4. Spray outlets 152 may include, for example, one or more of spray outlet 154, spray outlet 156, spray outlet 158, and/or spray outlet 160. Spray outlet 154, 156, 158, 160 may be configured to discharge fluid spray F1, F2, F3, F4 comprising a fluid when apparatus 100 (e.g., fitting 116) receives the fluid. In examples, apparatus 100 is configured to deliver the fluid from fitting 116 (e.g., fluid chamber 150) to spray outlet 154, 156, 158, 160. In some examples, spray outlet 154, 156, 158, 160 includes a nozzle 162 configured to generate one of fluid spray F1, F2, F3, F4 when spray outlet 154, 156, 158, 160 receives the fluid. Any number of spray outlets 152 may include a nozzle configured similarly to nozzle 162.

In examples, apparatus 100 is configured to direct fluid spray F1, F2, F3, F4 from spray outlet 154, 156, 158, 160 toward longitudinal axis L. Apparatus 100 may be configured to cause a fluid comprising fluid spray F1, F2, F3, F4 to impact a medical device (e.g., attachment member 102) within inner chamber 136 when spray outlet 154, 156, 158, 160 delivers fluid spray F1, F2, F3, F4. In examples, apparatus 100 is configured to cause the fluid comprising fluid spray F1, F2, F3, F4 to impact the medical device when the medical is inserted such that longitudinal axis L intersects at least some portion of the medical device. Apparatus 100 may be configured such that the impact (and, e.g., the kinetic energy) of fluid spray F1, F2, F3, F4 tends to cause fluid spray F1, F2, F3, F4 to displace (e.g., remove) matter collected by and/or adhering to at least attachment member 102. In examples, apparatus 100 is configured to increase a kinetic energy of fluid spray F1, F2, F3, F4 when a pressure of a fluid within fluid chamber 150 increases. Hence, when fluid delivery device 118 (FIG. 1) acts to increase a pressure of a fluid in device chamber 121 (FIG. 1) and provide the fluid to fluid chamber 150, apparatus 100 may be configured to respond by increasing the kinetic energy of fluid spray F1, F2, F3, F4.

As used here, when apparatus 100 delivers and/or otherwise provides fluid spray F1, F2, F3, F4, this means apparatus 100 delivers and/or otherwise provides at least one of fluid spray F1, fluid spray F2, fluid spray F3, and/or fluid spray F4 (e.g., using a fluid received via fitting 116). Spray outlet 154, 156, 158, 160 may indicate at least one of spray outlet 154, spray outlet 156, spray outlet 158, and/or spray outlet 160. Further, when spray outlet 154, 156, 158, 160 delivers fluid spray F1, F2, F3, F4, this means at least one of spray outlet 154, spray outlet 156, spray outlet 158, and/or spray outlet 160 delivers at least one of fluid spray F1, fluid spray F2, fluid spray F3, and/or fluid spray F4.

Apparatus 100 includes one or more fluid channels 164 (“fluid channels 164”) (FIG. 6) configured to fluidically couple fitting 116 (e.g., fluid chamber 150) and spray outlet 154, 156, 158, 160. Fluid channels 164 may include, for example, one or more of a fluid channel 166 and/or a fluid channel 168. In some examples, apparatus 100 is configured such that each of spray outlets 152 (e.g., each of spray outlet 154, spray outlet 156, spray outlet 158, and/or spray outlet 160) is fluidically coupled to fluid chamber 150 by at least one of fluid channels 164. For example, fluid channel 166 may be configured to fluidically couple spray outlet 154 and fluid chamber 150. Fluid channel 168 may be configured to fluidically couple spray outlet 160 and fluid chamber 150. Thus, when fluid chamber 150 receives a fluid (e.g., from fluid delivery device 118 (FIG. 1)), fluid channel 166 may be configured to direct a first portion of the fluid from fluid chamber 150 to spray outlet 154 to cause spray outlet 154 to issue fluid spray F1. Fluid channel 168 may be configured to direct a second portion of the fluid from fluid chamber 150 to spray outlet 160 to cause spray outlet 160 to issue fluid spray F4. Fluid channels 164 may be configured to direct other portions of the fluid to other spray outlets (e.g., spray outlet 156, spray outlet 158, and/or other spray outlets) to cause the other sprays outlet to issue other fluid sprays (e.g., fluid spray F2, fluid spray F3, and/or other fluid sprays).

For example, in some examples, fitting 116 includes a wall 170 defining an inner surface 172 (“fitting inner surface 172”) and an outer surface 174 (“fitting outer surface 174”) opposite fitting inner surface 172. Fitting inner surface 172 may define at least some portion of a boundary of fluid chamber 150. In examples, fitting inner surface 172 defines one or more fluid inlets which open to fluid channels 164, such as fluid inlet 176 and/or fluid inlet 178. Fluid channels 164 may be configured to receive the fluid from fluid chamber 150 via the one or more fluid inlets. For example, fluid channel 166 may be configured to receive the first portion of the fluid from fluid chamber 150 via fluid inlet 176 and/or fluid channel 168 may be configured to receive the second portion of the fluid from fluid chamber 150 via fluid inlet 178. In examples, wall 170 defines a fitting inlet 151 which defines an entry into fluid chamber 150. Fitting inlet 151 may be configured to receive fluid delivery device 118 (FIG. 1). In examples, fluid chamber 150 is configured to receive the fluid from fluid delivery device 118 when fluid delivery device is inserted through and/or coupled to fitting inlet 151 (e.g., by a clinician).

In some examples, fitting inner surface 172 defines a fluid inlet (e.g., similar to fluid inlet 176 and/or fluid inlet 178) for each fluid channel of fluid channels 164, although this is not necessarily required. In some examples, a fluid inlet (e.g., fluid inlet 176) may be configured to provide a portion of the fluid to a plurality of fluid channels (e.g., fluid channel 166, fluid channel 168, and/or other fluid channels of fluid channels 164). For example, in some examples, fitting inner surface 172 might define a single fluid inlet (e.g. one of fluid inlet 176 or fluid inlet 178), and fluid channels 164 may be configured to direct separate portions of the fluid to spray outlet 154, 156, 158, 160 at a location downstream of the single fluid inlet (e.g., when the fluid flows from fluid chamber 150 through the single fluid inlet in a downstream direction). Fitting inner surface 172 may define any number of fluid inlets configured to provide at least some portion of a fluid in fluid chamber 150 to fluid channels 164.

In some examples, apparatus housing 108 defines at least some portion of fluid channels 164. For example, apparatus housing 108 may be configured such that wall 138 defines at least some portion of a fluid channel (e.g., fluid channel 166 and/or fluid channel 168). In some examples, apparatus housing 108 is configured such that wall 138 defines substantially an entirety of a fluid channel extending from a fluid inlet (e.g., fluid inlet 176) to a spray outlet (e.g., spray outlet 154), however this is not required. In some examples, at least some portion of a fluid channel may be defined by a conduit fluidically coupling fluid chamber 150 and spray outlet 154, 156, 158, 160. In some examples, the conduit extends from apparatus housing 108 to a fitting configured to receive the fluid.

For example, FIG. 7 illustrates apparatus 200 including apparatus housing 208 and a fitting 216. Apparatus 200 includes wall 238 and one of more fluid channels 264, including a fluid channel 266 and a fluid channel 268. Fitting 216 includes fitting wall 270 defining fluid chamber 150 and fitting outer surface 274. Apparatus 200, apparatus housing 208, wall 238, fitting 216, fitting wall 270, fluid channels 264, fluid channel 266, fluid channel 268, and fitting outer surface 274 may be examples of apparatus 100, apparatus housing 108, wall 138, fitting 116, fitting wall 170, fluid channels 164, fluid channel 166, fluid channel 168, and fitting outer surface 174 respectively.

In examples, fitting 216 is substantially separable from apparatus housing 208. For example, apparatus 200 may be configured such that fitting 216 (e.g., fitting wall 270) may move (e.g., be moved by a clinician) independently of apparatus housing 208. Apparatus 200 may include one or more conduits 280 (“conduits 280”) including at least one conduit such as a conduit 282 and/or a conduit 284. Conduits 280 may extend from fitting 216 to apparatus housing 208. In examples, conduits 280 are mechanically coupled to fitting 216 and/or apparatus housing 208. In some examples, conduits 280 are substantially flexible conduits, such that fitting 216 may move independently of apparatus housing 208 (e.g., when fitting 216 is moved and/or handled by a clinician).

Conduits 280 may define at least some portion of fluid channels 264. For example, conduit 282 may define at least a portion of fluid channel 266. Conduit 284 may define at least a portion of fluid channel 268. Hence, in some examples, apparatus 200 may be configured such that each of spray outlets 152 (FIGS. 3-6) is fluidically coupled to fluid chamber 150 by conduits 280. For example, conduit 282 (defining at least a portion of fluid channel 266) may be configured to fluidically couple spray outlet 154 and fluid chamber 150. Conduit 284 (defining at least a portion of fluid channel 268) may be configured to fluidically couple spray outlet 160 and fluid chamber 150. Thus, when fitting 216 (e.g., fluid chamber 150) receives a fluid (e.g., from fluid delivery device 118 (FIG. 1)), conduit 282 may be configured to direct a portion of the fluid to spray outlet 154 to cause spray outlet 154 to issue fluid spray F1. Conduit 284 may be configured to direct another portion of the fluid to spray outlet 160 to cause spray outlet 160 to issue fluid spray F4. Conduits 280 may be configured to direct other portions of the fluid to other spray outlets (e.g., spray outlet 156, spray outlet 158, and/or other spray outlets) to cause the other sprays outlet to issue other fluid sprays (e.g., fluid spray F2, fluid spray F3, and/or other fluid sprays). Further, conduits 280 may include any number of conduits configured similarly to conduit 282, 284. For example, in some examples, conduits 280 may consist of a single conduit extending between fitting 216 and apparatus housing 208.

In some examples, apparatus housing 108, 208 may substantially define spray outlet 154, 156, 158, 160. For example, spray outlet 154, 156, 158, 160 may substantially be an orifice or a nozzle defined by apparatus housing 108, 208 (e.g., wall 138, 238). The orifice or the nozzle may be configured to issue fluid spray F1, F2, F3, F4 when fluid channels 164, 264 deliver a fluid to the orifice or nozzle. In some examples, spray outlet 154, 156, 158, 160 may be a component supported by (e.g., coupled to) apparatus housing 108, 208 (e.g., wall 138, 238). For example, spray outlet 154, 156, 158, 160 may be an individual component is separated from and/or separable from apparatus housing 108, 208 (e.g., wall 138, 238) until spray outlet 154, 156, 158, 160 is affixed to apparatus housing 108, 208 (e.g., wall 138, 238). In some examples, spray outlet 154, 156, 158, 160 may be supported by an inner conduit (e.g., inner conduit 224 (FIG. 9)) extending between apparatus housing 108, 208 (e.g., wall 138, 238) and spray outlet 154, 156, 158, 160.

Magnification pane 112 is configured to allow visual observation (e.g., by a clinician) of attachment member 102 when attachment member 102 is positioned within inner chamber 136. Magnification pane 112 may be configured to enlarge the apparent size of attachment member 102 within inner chamber 136, such that the clinician may evaluate an effectiveness of fluid spray F1, F2, F3, F4 in the removal of matter from attachment member 102. Apparatus housing 108, 208 is configured to support magnification pane 112 such that a field-of-view of magnification pane 112 includes at least a portion of inner chamber 136 into which attachment member 102 is expected to extend when attachment member 102 and/or medical device distal portion 103 are inserted into inner chamber 136 via entry passage 110.

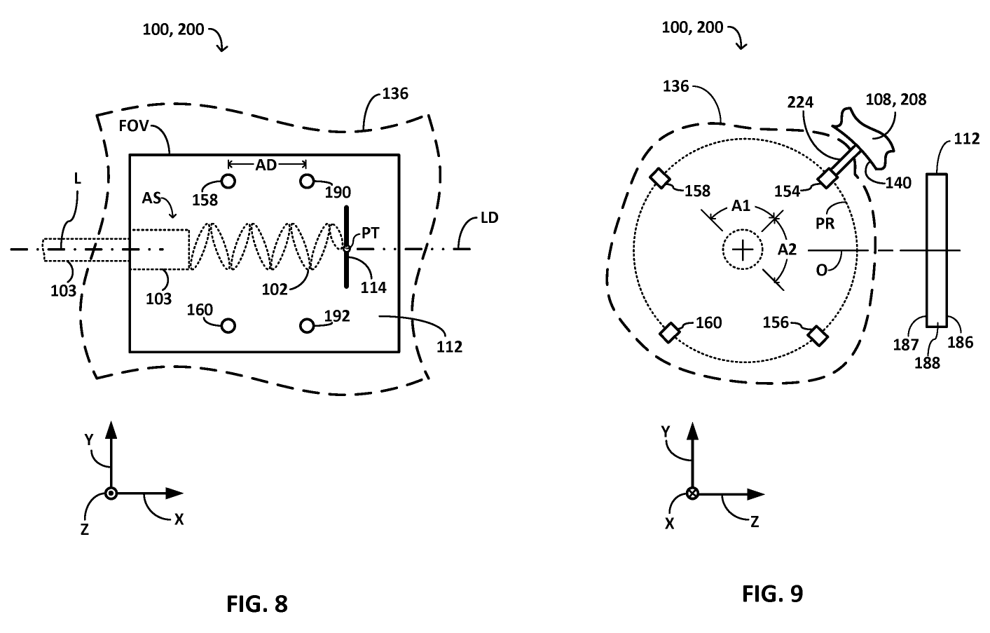

For example, FIG. 8 is a schematic plan view depicting magnification pane 112 and a portion of inner chamber 136 illustrated in accordance with the X-Y-Z axis shown, with the Z axis proceeding out of the page. FIG. 9 is a schematic end view of depicting magnification pane 112 and the portion of inner chamber 136 illustrated in accordance with the X-Y-Z axis shown, with the X axis proceeding into the page. FIG. 8 and FIG. 9 also depict medical device housing 104 and attachment member 102 with dashed lines and extending into the portion of inner chamber 136, for reference.

Magnification pane 112 defines a field-of-view (FOV) which includes at least a portion of inner chamber 136. In examples, the FOV is defined by a boundary of magnification pane 112. Magnification pane 112 is configured to enlarge an apparent size AS of an object (e.g., to provide an apparent image of the object) such as attachment member 102 and/or medical device housing 104 when the object (e.g., attachment member 102 and/or medical device housing 104) is positioned in the portion of inner chamber 136 within the FOV of magnification pane 112. Magnification pane 112 may be supported by apparatus housing 108, 208 such that longitudinal axis L extends through the portion of the portion of inner chamber 136 within the FOV. In examples, magnification pane 112 defines a convex lens. In examples, apparatus housing 108, 208 supports magnification pane 112 such that magnification pane 112 defines an optical axis O which extends through inner chamber 136. In some examples, apparatus housing 108, 208 supports magnification pane 112 such that optical axis O is substantially perpendicular to longitudinal axis L. In some example, magnification pane is configured to provide at least a 2X magnification, in some examples at least a 5X magnification, and in some examples at least a 10X magnification of an object within the FOV.

In some examples, magnification pane 112 includes visual marker 114 configured to appear within the FOV of magnification pane 112. In examples, magnification pane 112 supports and/or defines visual marker 114 (e.g., supports visual marker 114 on a surface 186 or surface 187 of magnification pane 112, or within a body 188 of magnification pane 112). In other examples, a portion of inner surface 140 (FIGS. 3-6) may support and/or define visual marker 114. Visual marker 114 may be configured to demarcate a boundary (e.g., a geometric plane) within inner chamber 136 to, for example, serve as an observable limit for the extension of a medical device (e.g., attachment member 102 and medical device distal portion 103) into inner chamber 136. For example, visual marker 114 may demarcate the boundary such that attachment member 102 positions within inner chamber 136 substantially at a location where fluid spray F1, F2, F3, F4 is expected to more efficiently and/or effectively impact the medical device, and/or such that contact between the medical device (e.g., attachment member 102) and apparatus housing 108, 208 is limited and/or avoided.

In examples, visual marker 114 is configured to provide an indication of a position PT on longitudinal axis L. For example, visual marker 114 may substantially demarcate a geometric plane passing through visual marker 114 and substantially perpendicular to longitudinal axis L (e.g., a plane extending into and out of the page in FIG. 8). Visual marker 114 may define position PT at an intersection of the geometric plane and longitudinal axis L. Hence apparatus 100, 200 may be configured such that a clinician may extend the medical device (e.g., attachment member 102 and/or medical device distal portion 103) into inner chamber 136 (e.g., via entry passage 110) while observing the medical device through the FOV of magnification pane 112.

Visual marker 114 may be configured to indicate to the clinician when it may be advantageous to cease the extension of the medical device into inner chamber 136, such that the medical device is substantially positioned at a location where fluid spray F1, F2, F3, F4 is expected to more efficiently and/or effectively impact the medical device, or such that contact between the medical device and apparatus housing 108, 208 is limited and/or avoided, and/or for other reasons. For example, visual marker 114 may be configured to indicate a portion of the medical device (e.g., a distal end of the medical device) is in proximity to point PT when the apparent image of the portion of the medical device provided by magnification pane 112 is substantially coincident with visual marker 114 or an apparent image of visual marker 114. A clinician may cease the extension of the medical device into inner chamber 136 when the apparent image of the portion of the medical device is substantially coincident with visual marker 114 or the apparent image of visual marker 114.

As discussed, apparatus 100, 200 may be configured to cause spray outlets 152 to direct fluid spray F1, F2, F3, F4 toward longitudinal axis L. In some examples, spray outlets 152 includes a plurality of fluid outlets arranged around a perimeter PR surrounding longitudinal axis L. Apparatus 100, 200 may be configured such that the plurality of fluid outlets direct fluid sprays F1, F2, F3, F4 toward longitudinal axis L along a plurality of radial directions. In examples, spray outlets 152 define one or more subtended angles with vertices on longitudinal axis L. For example, spray outlet 154 and spray outlet 158 may be arranged (e.g., on perimeter PR) to subtend a first angle A1 having a first vertex on longitudinal axis L. Spray outlet 154 and spray outlet 156 may be arranged (e.g., on perimeter PR) to subtend a second angle A2 having a second vertex on longitudinal axis L. Spray outlets 152 may be configured to subtend additional angles over a full 360 degrees around longitudinal axis L. Further, although perimeter PR is depicted as a substantially circular perimeter defined within the Y-Z plane, this is not required. Perimeter PR may define any closed curve surrounding longitudinal axis L, and may extend in the X direction, the Y direction, and the Z direction.

In some examples, instead of or in addition to the arrangement of spray outlets 152 arranged around perimeter PR, spray outlets 152 may be axially distributed, such that spray outlets 152 direct fluid sprays F1, F2, F3, F4 over a length of longitudinal axis L. For example, spray outlets 152 may include one or more spray outlets such as spray outlet 190 and/or spray outlet 192. Spray outlet 190 may be axially displaced (e.g., in the X direction) from spray outlet 158 by a displacement AD. Spray outlet 190 may be displaced by the displacement AD such that spray outlet 158 substantially distributes a first fluid spray over a first portion of a medical device (e.g., medical device distal portion 103 and/or attachment member 102) as spray outlet 190 distributes a second fluid spray over a second portion of the medical device longitudinally displaced from the first portion. Spray outlet 192 may be similarly displaced from spray outlet 160 (e.g., by displacement AD or another displacement). Further, spray outlet 190, spray outlet 192, and other spray outlets may be arranged around a perimeter surrounding longitudinal axis L in a similar manner to that described for spray outlets 154, 156, 158, 160 around perimeter PR.

Referring primarily to FIG. 5, in some examples, apparatus housing 108, 208 includes an entry portion 194 and an end portion 196. Apparatus housing 108, 208 may be configured such that inner chamber 136 separates (e.g., is between) entry portion 194 and end portion 196. In examples, entry passage 110 extends through entry portion 194. In some examples, entry portion 194 defines entry inlet 144 and/or entry outlet 146. In examples, visual marker 114 is configured to define point PT (FIG. 8) between entry portion 194 and end portion 196.

In some examples, entry portion 194 defines an entry surface 202. Entry surface 202 may be a portion of inner surface 140. In examples, entry surface 202 defines some portion of boundary B surrounding inner chamber 136. In some examples, entry inlet 144 opens to entry surface 202. End portion 196 may define an end surface 198. In examples, end surface 198 substantially faces entry surface 198. End surface 198 may be another portion of inner surface 140. In examples, end surface 198 defines another portion of boundary B. In some examples, visual marker 114 is configured to define point PT (FIG. 8) between entry surface 202 and end surface 198. In some examples, guide portion 135 and guide surface 137 substantially extend from entry surface 202 (e.g. toward end portion 196).

In some examples, end surface 198 substantially faces entry outlet 146. End surface 198 may be configured to deform (e.g., to elastically deform) when a force (e.g., substantially parallel to longitudinal axis L) is imparted to end surface 198. For example, end surface 198 may be configured to deform if a distal end of a medical device (e.g., attachment member 102) contacts and imparts the force to end surface 198. End surface 198 may be configured to deform to, for example, mitigate and/or avoid deformation and/or damage to attachment member 102 should attachment member 102 contact end surface 198 (e.g., during extension of medical device distal portion 103 and attachment member 102 into inner chamber 136). In some examples, instead of or in additional to visual marker 114, end surface 198 may be configured to assist in positioning attachment member 102 within inner chamber 136. For example, end surface 198 may be configured such that contact between attachment member 102 and end surface 198 places attachment member 102 in a position (e.g., a position along longitudinal axis L) where fluid spray F1, F2, F3, F4 might be expected to more efficiently impact attachment member 102. End surface 198 may be configured to deform to substantially “give” when attachment member 102 contacts end surface 198.

In some examples, apparatus housing 108, 208 is configured to drain fluid introduced to inner chamber 136 by fluid spray F1, F2, F3, F4 from inner chamber 136. For example, apparatus housing 108, 208 may define a drain passage 204 configured to drain the fluid (e.g., by gravity) to a reservoir 206 defined by housing 108, 208. In some examples, apparatus housing 108, 208 defines an upper housing 210 and a tray portion 212. Tray portion 212 may be configured to be separable from upper housing 210. Tray portion 212 may be configured to define reservoir 206. Thus, apparatus housing 108, 208 may be configured such the fluid of fluid spray F1, F2, F3, F4 and/or matter removed from attachment member 102 may exit inner chamber 136 to reservoir 206 via drain passage 204. Apparatus housing 108, 208 may be configured such that a clinician may separate upper housing 210 and tray portion 212 for, for example, removal and/or disposal of the fluid of fluid spray F1, F2, F3, F4 and/or the matter removed.

As discussed, apparatus housing 108, 208 may be configured to position within a boundary of a sterile field (e.g., a sterile space) established and maintained during an implantation procedure. In examples, apparatus housing 108, 208 has a size and/or weight configured to allow a clinician to handle, place, and/or relocate apparatus 108, 208 within the sterile field. In some examples, apparatus 108, 208 is configured to occupy a volume of less than about 1000 in³ (inches cubed) (16.38 liters), in some examples configured to occupy a volume of less than about 216 in³ (3.54 liters). In some examples, apparatus 108, 208 is configured to fit within a 10-inch (254 millimeter) by 10-inch by 10-inch cubic volume, in some examples configured to fit within a 6-inch (152 millimeter) by 6-inch by 6-inch cubic volume. In some examples, at least upper housing 210 and tray portion 212 in combination are configured to occupy a volume of less than about 1000 in³ in some examples and/or less than about 216 in³ in some examples, and/or configured to fit within a 10-inch by 10-inch by 10-inch cubic volume in some examples and/or fit within a 6-inch by 6-inch by 6-inch cubic volume in some examples.

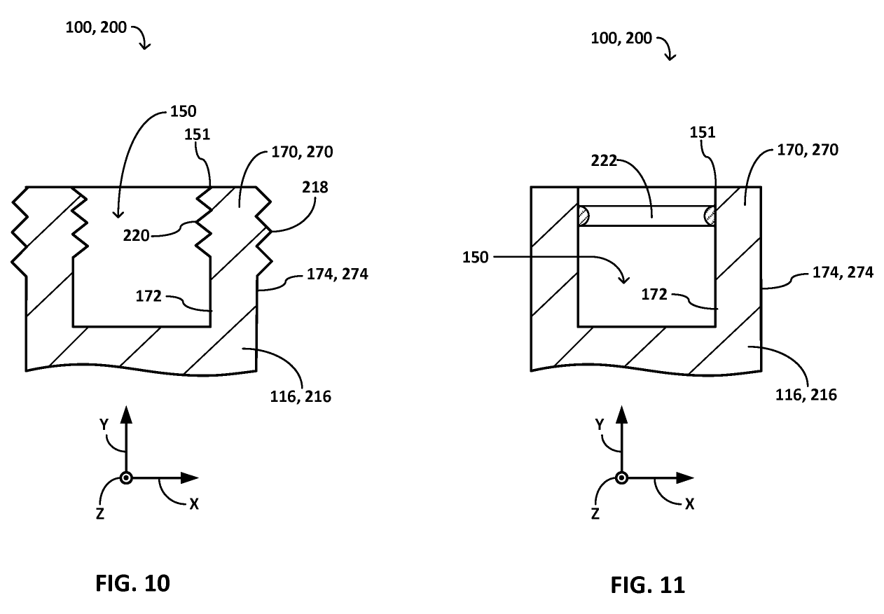

Fitting 116, 216 may be configured to establish a substantially fluid-tight seal with fluid delivery device 118 when fluid delivery device 118 delivers a fluid to fluid chamber 150. For example, FIG. 10 is a schematic cross-sectional view of a portion of an example fitting 116, 216 taken with cutting plane A-A' of FIG. 4 and illustrated in accordance with the X-Y-Z axis shown, with the Z axis proceeding out of the page. FIG. 11 is a schematic cross-sectional view of a portion of another example fitting 116, 216 taken with cutting plane A-A' of FIG. 4 and illustrated in accordance with the X-Y-Z axis shown, with the Z axis proceeding out of the page.

Fitting 116, 216 may be configured to mechanically engage and/or mechanically couple with fluid delivery device 118 to, for example, establish the substantially fluid-tight seal, secure the fluid delivery device in a condition to provide fluid to fluid chamber 150, or for other reasons. For example, as depicted in FIG. 10, fitting 116, 216 may be configured to threadably engage fluid delivery device 118. In examples, fitting 116, 216 (e.g., fitting outer surface 174, 274) defines a set of threads (e.g., external threads 218) configured to engage fluid delivery device 118. In some examples, fitting 116, 216 (e.g., fitting inner surface 172) defines a set of threads (e.g., internal threads 220) configured to engage fluid delivery device 118. Fitting 116, 216 may define external threads 218 and/or internal threads 220 either alone or in combination.

In some examples, for example as depicted in FIG. 11, fitting 116, 216 may be configured to mechanically engage (e.g., frictionally engage) fluid delivery device 118 with a one or more protrusions in order to establish the substantially fluid-tight seal, secure the fluid delivery device in a condition to provide fluid to fluid chamber 150, or for other reasons. For example, fitting 116, 216 may include an protrusion element 222 such as an O-ring configured to protrude from fitting inner surface 172. Protrusion element 222 may be configured to engage fluid delivery device 118 when fluid delivery device 118 inserts into fluid chamber 150. In examples, protrusion element 222 is a compressible element configured to compress when fluid delivery device 118 inserts into fluid chamber 150. In some examples, fitting 116, 216 may be configured to mechanically engage and/or mechanically couple with fluid delivery device 118 in any manner to establish the substantially fluid-tight seal, secure the fluid delivery device in a condition to provide fluid to fluid chamber 150, or for other reasons. For example, in some examples, fitting 116, 216 may include a quick connect fitting (e.g., a push fitting) configured to mechanically engage and/or mechanically couple with fluid delivery device 118, or include some other type of engagement component.

In examples, processing circuitry 124 of medical system 106 may include fixed function circuitry and/or programmable operating circuitry. In examples, processing circuitry 124 includes circuitry configured to perform one or more functions of operating circuitry, such as therapy delivery circuitry, sensing circuitry, processing circuitry, switching circuitry, communication circuitry, and/or other circuitries. Processing circuitry 124, as well as other processors, operating circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 124 includes multiple components, such as any combination of one or more microprocessors, one or more DSPs, one or more ASICs, or one or more FPGAs, as well as other discrete or integrated logic circuitry, and/or analog circuitry.

Functions attributed to processing circuitry 124 may be embodied as software, firmware, hardware or any combination thereof. Processing circuitry 124 may include, for instance, a variety of capacitors, transformers, switches, and the like configured to perform the functions of processing circuitry 124. In examples, processing circuitry 124 may be configured to communicate with another device, such as a patient input/output device, a clinician input/output device, and/or others. Processing circuitry 124 may include any suitable hardware, firmware, software or any combination thereof for communicating with another device. In addition, processing circuitry 124 may communicate with a networked computing device and a computer network. In examples, processing circuitry 124 and/or other circuitry of medical system 106 is configured to deliver stimulation signals to and/or receive sensing signals from electrode 109, other electrodes supported by medical device housing 104 or attachment member 102, and/or other electrodes and/or sensors within medical system 106 or external to medical system 106. Processing circuitry 124 may be configured to provide electrical signals, e.g., pacing therapy, electrode 109, other electrodes supported by medical device housing 104 or attachment member 102, and/or other electrodes and/or sensors within medical system 106 or external to medical system 106. Processing circuitry 124 may be configured to receive electrical signals, e.g., sensed cardiac electrical signals, from electrode 109, other electrodes supported by medical device housing 104 or attachment member 102, and/or other electrodes and/or sensors within medical system 106 or external to medical system 106.

Medical system 106 (e.g., processing circuitry 124) can also include a memory configured to store program instructions, such as software, which may include one or more program modules, which are executable by processing circuitry 124. The program instructions may be embodied in software and/or firmware. The memory can include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), flash memory, or any other digital media. In some examples, the memory includes computer-readable instructions that, when executed by processing circuitry 124 cause processing circuitry 124 to perform various functions described herein and/or other functions of processing circuitry 124.

Medical device housing 104 may be configured to fluidly isolate conductor 125 and/or processing circuitry 124 and/or other circuitry from an environment in contact with an exterior surface of medical device housing 104. In examples, medical device housing 104 is configured to hermetically seal at least an enclosure defined by medical device housing 104 and holding conductor 125. Medical device housing 104 (e.g., medical device distal portion 103) and/or attachment member 102 may be configured to define shapes that are easily accepted by the patient's body while minimizing patient discomfort. For example, portions of medical device housing 104 (e.g., medical device distal portion 103 and/or medical device proximal portion 105) may define a substantially cylindrical shape with cylindrical sidewalls. In other examples, portions of medical device housing 104 may define substantially rectangular or other non-cylindrical shapes. medical device housing 104 may define shapes in which corners and edges are designed with relatively large radii, in order to present a housing having smoothly contoured exterior surfaces.

As used here, when a first portion of a system (e.g., medical system 106) is substantially parallel to a second portion of or an axis defined by the system, this may mean the first portion is parallel or nearly parallel to the second portion or the axis to the extent permitted by manufacturing tolerances. In some examples, when the first portion is substantially parallel to the second portion or the axis, this may mean a first vector defined by the first component of the system defines an angle of less than 10 degrees, in some examples less than 5 degrees, and in some examples less than 1 degree, with a second vector defined by the second component or the axis. When a first portion of the system is substantially perpendicular to a second portion of or an axis defined by the system, this may mean the first portion is perpendicular or nearly perpendicular to the second portion or the axis to the extent permitted by manufacturing tolerances. In some examples, when the first portion is substantially perpendicular to the second portion or the axis, this may mean that the first vector defined by the first component of the system defines an angle of at least 80 degrees, in some examples at least 85 degrees, and in some examples at least 89 degrees, with the second vector defined by the second component.

As used here, when a first portion of a system (e.g., medical system 106) supports a second portion of the system, this means that when the second portion causes a first force to be exerted on the first portion, the first portion causes a second force to be exerted on the second portion in response to the first force. The first force and/or second force may be a contact force and/or an action-at-a-distance force. For example, first force and/or second force may be mechanical force, a magnetic force, a gravitational force, or some other type of force. The first portion of the system may be a portion of the system or a portion of a component of the system. The second portion of the system may be another portion of the system or another portion of the same component or a different component. In some examples, when the first portion of the system supports the second portion of the system, this may mean the second portion is mechanically supported by and/or mechanically connected to the first portion.

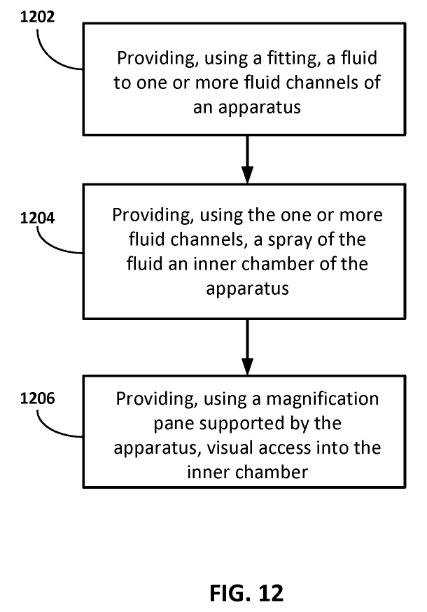

A technique for providing a fluid spray into an inner chamber of an apparatus is illustrated in FIG. 12. Although the technique is described mainly with reference to medical system 106 of FIGS. 1-11, the technique may be applied to other medical systems in other examples.

The technique includes providing, using a fitting 116, 216 of apparatus 100, 200, a fluid to fluid channels 164, 264 (1202). Apparatus housing 108, 208 may support and/or define at least some portion of fluid channels 164, 264. Fitting 116, 216 may receive the fluid within a fluid chamber 150 defined by a wall 170, 270 to fitting 116, 216. In examples, fitting 116, 216 receives the fluid from a fluid delivery device 118. In examples, fluid delivery device 118 pressurizes the fluid as fluid delivery device 118 provides the fluid to fitting 116, 216. Fluid delivery device 118 may pressurize the fluid by moving (e.g., by a clinician) a plunger 117 relative to a barrel 119 to pressurize the fluid within a device chamber 121.

In examples, fitting 116, 216 couples (e.g., mechanically couples) as fluid delivery device 118 provide the fluid. Fitting 116, 216 may provide a substantially fluid-type seal with fluid delivery device 118 when fitting 116, 216 couples with fluid delivery device 118. In some examples, fitting 116, 216 threadably engages fluid delivery device 118 (e.g., using external threads 218 and/or internal threads 220) to couple with fluid delivery device 118. On some examples, fitting 116, 216 engages fluid delivery device 118 using a protrusion element 222, such as n O-ring or quick connect type fitting.

The technique includes providing, using fluid channels 164, 264, a fluid spray F1, F2, F3, F4 to an inner chamber 136 defined by apparatus housing 108, 208 (e.g., defined by an inner surface 140) (1204). In examples, apparatus 100, 200 causes fluid spray F1, F2, F3, F4 to impact a medical device (e.g., attachment member 102 and/or medical device distal portion 103) within inner chamber 136. In examples, the medical device is inserted into inner chamber 136 through an entry passage 110 extending through a wall 138 of apparatus housing 108, 208 from an outer surface 142 to inner surface 140. In examples, apparatus 100, 200 provides fluid spray F1, F2, F3, F4 using spray outlets 152.

The technique may include removing tissue and/or other anatomical matter from the medical device (e.g., attachment member 102) using fluid spray F1, F2, F3, F4. In examples, fluid spray F1, F2, F3, F4 removes the tissue and/or other anatomical matter from the medical device following implantation of attachment member 102 within a tissue wall of an anatomical volume (e.g., a heart chamber) of a patient. In some examples, fluid spray F1, F2, F3, F4 removes the tissue and/or other anatomical matter subsequent to implantation of attachment member 102 at a first tissue location P1 and prior to implantation of attachment member 102 at a second tissue location P2.

The technique includes providing, using a magnification pane 112 supported by apparatus housing 108, 208, visual access to a portion of inner chamber 136 (1206). In examples, magnification pane 112 provides the visual access through a field-of-view (FOV) defined by magnification pane 112. In examples, the FOV includes at least some portion of a longitudinal axis L defined by apparatus housing 108, 208 and extending through inner chamber 136. In some examples, magnification pane 112 provides an apparent image (e.g., a magnified image) of the medical device (e.g., medical device distal portion 103 and/or attachment member 102) when the medical device is positioned within inner chamber 136.

In examples, the technique includes positioning the medical device within inner chamber 136 using a visual marker 114 visible within the FOV of magnification pane 112. The technique may include inserting the medical device through entry passage 110 until an apparent image of some portion of the medical device (e.g., a distal end of attachment member 102) is in proximity to and/or substantially coincident with an apparent or actual image of visual marker 114. In some examples, body 188 and/or surface 186, 187 of magnification pane 112 supports visual marker 114. In some examples, a portion of inner surface 140 (e.g., visible within the FOV) supports visual marker 114.

In some examples, fluid of fluid spray F1, F2, F3, F4 drains from inner chamber 136 through a drain passage 204 defined by housing apparatus 108, 208. The fluid may drain (e.g., via drain passage 204) from inner chamber 136 to a reservoir 206 defined by apparatus housing 108, 208. In examples, an upper housing 210 defines drain passage 204 and/or inner chamber 136. In examples, a tray portion 212 defines reservoir 206. In examples, the technique includes separating upper housing 210 and tray portion 212. In some examples, the technique includes displacing the fluid of fluid spray F1, F2, F3, F4 and/or matter moved from the medical device (e.g., attachment member 102) by fluid spray F1, F2, F3, F4 from upper housing 210 and/or inner chamber 136 by displacing (e.g., by a clinician) tray portion 212 from upper housing 210.

Various examples of the disclosure have been described. Any combination of the described systems, operations, or functions is contemplated. These and other examples are within the scope of the following claims.

Various aspects of the techniques may enable the following examples.

Example 1. An apparatus, comprising: a housing, the housing defining an outer surface and an inner surface opposite the outer surface, the inner surface defining an inner chamber, and the housing defining a longitudinal axis extending through the inner chamber, wherein: the housing defines an entry passage opening into the inner chamber and intersected by the longitudinal axis, the entry passage extending from the outer surface to the inner surface, the housing supports one or more fluid channels, each fluid channel fluidly coupled to a spray outlet which opens into the inner chamber, and each spray outlet configured to provide a spray of a fluid into the inner chamber when the one or more fluid channels receives the fluid, and the housing supports a magnification pane configured to provide visual access through the housing and into the inner chamber; and a fitting defining a fluid chamber configured to hold the fluid, wherein the fluid chamber is configured to provide the fluid to the one or more fluid channels.

Example 2. The apparatus of claim 1, wherein the housing supports the magnification pane to cause a field of view of the magnification pane to encompass a portion of the inner chamber through which the longitudinal axis extends.

Example 3. The apparatus of claim 1 or claim 2, wherein the magnification pane comprises a convex lens.

Example 4. The apparatus of any of claims 1-4, wherein the magnification pane defines an optical axis intersecting the inner chamber.

Example 5. The apparatus of claim 4, wherein the optical axis is substantially perpendicular to the longitudinal axis.

Example 6. The apparatus of any of claims 1-5, wherein the magnification pane defines a visual marker configured to define a location within the field of view of the magnification pane.

Example 7. The apparatus of claim 6, wherein the housing includes an entry portion and an end portion substantially facing the entry portion, wherein the entry portion defines the entry passage, and wherein visual marker defines the location on the longitudinal axis and between the entry portion and the end portion.

Example 8. The apparatus of any of claims 1-7, wherein the end portion defines an end surface substantially facing the entry portion, and wherein the end surface is configured to elastically deform in response to a force imparted substantially parallel to the longitudinal axis.

Example 9. The apparatus of any of claims 1-8, wherein the housing defines a guide surface extending from the entry portion, wherein the guide surface extends in a direction substantially parallel to or coincident with the longitudinal axis.

Example 10. The apparatus of any of claims 1-9, wherein the fitting defines a fitting opening which opens into the fluid chamber, wherein the opening is configured to receive a device, and wherein the fluid chamber is configured to receive the fluid from the device when the fitting opening receives the device.

Example 11. The apparatus of claim 10, wherein the fitting is configured to engage the device to form a fluid-tight seal with the device when the fitting opening receives the device.

Example 12. The apparatus of claim 11, wherein the fitting includes an o-ring surrounding the opening, wherein the o-ring is configured to form the fluid-tight seal with the device.

Example 13. The apparatus of claim 11 or claim 12, wherein the fitting is configured to threadably engage the device.

Example 14. The apparatus of any of claims 10-13, further comprising the device, wherein the device is configured to pressurize the fluid in the fluid chamber when the fluid chamber receives the fluid from the device, and wherein the fitting is configured to provide the fluid to the one or more fluid channels when the device pressurizes the fluid in the fluid chamber.

Example 15. The apparatus of claim 14, wherein the device is a syringe.

Example 16. The apparatus of any of claims 1-15, wherein the outer surface of the housing supports the fitting.

Example 17. The apparatus of any of claims 1-16, wherein the fitting includes an inner fitting surface defining the fluid chamber and an outer fitting surface opposite the inner fitting surface, wherein the outer fitting surface is contiguous with the outer surface of the housing.

Example 18. The apparatus of any of claims 1-17, wherein the fitting supports one or more fluid channel openings which open into the fluid chamber, wherein the one or more fluid channel openings are fluidly coupled to the one or more fluid channels.

Example 19. The apparatus of claim 18, wherein the fitting supports a plurality of fluid channel openings, wherein the one or more fluid channels fluidly couple each fluid channel opening with at least one spray outlet.

Example 20. The apparatus of any of claims 1-19, wherein each spray outlet includes a nozzle configured to generate the spray of a fluid when the one or more fluid channels receives the fluid.

Example 21. The apparatus of any of claims 1-20, wherein the each spray outlet is configured to direct the spray of the fluid toward the longitudinal axis when the each spray outlet provides the spray of the fluid into the inner chamber.

Example 22. The apparatus of claim 21, wherein the each spray outlet is configured to cause the spray of the fluid to intersect the longitudinal axis when the each spray outlet provides the spray of the fluid into the inner chamber.

Example 23. The apparatus of any of claims 1-22, wherein the one or more fluid channels define a plurality of spray outlets which include the each spray outlet, and wherein the plurality of spray outlets substantially surround the longitudinal axis.

Example 24. The apparatus of any of claims 1-23, wherein the entry passage is configured to receive an implantable medical lead, and wherein the each spray outlet is configured to direct the spray of a fluid toward an attachment member positioned within the inner chamber when the entry passage receives the implantable medical lead.

Example 25. The apparatus of claim 24, further comprising the implantable medical lead, wherein the implantable medical lead includes a medical device distal portion defining a medical device distal end, wherein the medical device distal portion supports the attachment member such that the attachment member extends distal to the medical device distal end.

Example 26. An apparatus, comprising: a housing, the housing defining an outer surface and an inner surface opposite the outer surface, the inner surface defining an inner chamber, and the housing defining a longitudinal axis extending through the inner chamber, wherein: the housing defines an entry passage intersected by the longitudinal axis, the entry passage extending from the outer surface to the inner surface, the housing supports a one or more fluid channels, each fluid channel defining a spray outlet which opens into the inner chamber, and each spray outlet configured to provide a spray of a fluid into the inner chamber when the one or more fluid channels receives the fluid, the housing supports a magnification pane configured to provide visual access through the housing and into the inner chamber, the housing supports the magnification pane to cause a field of view of the magnification pane to encompass a portion of the inner chamber through which the longitudinal axis extends, and the magnification pane defines an optical axis substantially perpendicular to the longitudinal axis; and a fitting defining a fluid chamber configured to hold the fluid, wherein the fluid chamber is configured to provide the fluid to the one or more fluid channels.

Example 27. The apparatus of claim 26, wherein the fitting defines a fitting opening which opens into the fluid chamber, wherein the opening is configured to receive a device, and wherein the fluid chamber is configured to receive the fluid from the device when the fitting opening receives the device.

Example 28. The apparatus of claim 26 or claim 27, wherein the magnification pane defines a visual marker, the visual marker configured to define a location within the field of view of the magnification pane and on the longitudinal axis when a vector substantially perpendicular to the longitudinal axis passes through the visual marker.

Example 29. A method, comprising: providing, using a fitting defining a fluid chamber, a fluid to a one or more fluid channels supported by a housing; providing, using the one or more fluid channels, at least one spray of a fluid into an inner chamber defined by an inner surface of the housing, wherein the housing defines a longitudinal axis extending through the inner chamber, wherein the housing defines an entry passage opening into the inner chamber and intersected by the longitudinal axis, and wherein the entry passage extends between the inner surface and an outer surface defined by the housing and opposite the inner surface; and providing, using a magnification pane supported by the housing, visual access through the housing and into the inner chamber.

Example 30. The method of claim 29, further comprising causing, using the magnification pane, a field of view to encompass a portion of the inner chamber through which the longitudinal axis extends.

Example 31. The method of claim 29 or claim 30, further comprising defining, using the magnification pane, an optical axis intersecting the inner chamber.

Example 32. The method of any of claims 29-31, further comprising defining, using the magnification pane, a focal length substantially equal to a pane displacement from the magnification pane to the longitudinal axis.

Example 33. The method of any of claims 29-32, further comprising defining, using a visual marker of the magnification pane, a location within the field of view of the magnification pane and on the longitudinal axis.

Example 34. The method of any of claims 29-33, further comprising: receiving, using a fitting opening of the fitting, a fluid delivery device; and receiving, using the fluid chamber, the fluid from the fluid delivery device.

Example 35. The method of claim 34, further comprising forming, using the fitting, a fluid-tight seal with the device when the fitting opening receives the device.

Example 36. The method of claim 34 or claim 35, further comprising pressurizing, using the device, the fluid in the fluid chamber.

Example 37. The method of any of claims 29-36, further comprising directing, using one of more spray outlets defined by the one or more fluid channels, the at least one spray of the fluid toward the longitudinal axis.

Example 38. The method of claim 37, further comprising intersecting the longitudinal axis with the at least one spray.

Example 39. The method of any of claims 29-38, further comprising surrounding, using a plurality of spray outlets defined by the one or more fluid channels, the longitudinal axis, wherein each spray outlet in the plurality of spray outlets is configured to direct a spray of the fluid toward the longitudinal access when the fitting provides the fluid to the one or more fluid channels.

Example 40. The apparatus of any of claims 29-39, further comprising: extending, using a medical device, the medical device through the entry passage to position an attachment member of the medical device within the inner chamber; and directing, using the each spray outlet, the spray of a fluid toward the attachment member.

Example 41. The method of claim 40, wherein the implantable medical device is an implantable medical lead.

Example 42. The method of claim 40 or claim 41, further comprising observing, using the magnification pane, the attachment member.

1. An apparatus, comprising:
a housing, the housing defining an outer surface and an inner surface opposite the outer surface, the inner surface defining an inner chamber, and the housing defining a longitudinal axis extending through the inner chamber, wherein:
the housing defines an entry passage opening into the inner chamber and intersected by the longitudinal axis, the entry passage extending from the outer surface to the inner surface,
the housing supports one or more fluid channels, each fluid channel fluidly coupled to a spray outlet which opens into the inner chamber, and each spray outlet configured to provide a spray of a fluid into the inner chamber when the one or more fluid channels receives the fluid, and
the housing supports a magnification pane configured to provide visual access through the housing and into the inner chamber; and
a fitting defining a fluid chamber configured to hold the fluid, wherein the fluid chamber is configured to provide the fluid to the one or more fluid channels.

2. The apparatus of claim 1, wherein the housing supports the magnification pane to cause a field of view of the magnification pane to encompass a portion of the inner chamber through which the longitudinal axis extends.

3. The apparatus of claim 1, wherein the magnification pane comprises a convex lens.

4. The apparatus of claim 1, wherein the magnification pane defines an optical axis intersecting the inner chamber.

5. The apparatus of claim 4, wherein the optical axis is substantially perpendicular to the longitudinal axis.

6. The apparatus of claim 1, wherein the magnification pane defines a visual marker configured to define a location within the field of view of the magnification pane.

7. The apparatus of claim 6, wherein the housing includes an entry portion and an end portion substantially facing the entry portion, wherein the entry portion defines the entry passage, and wherein visual marker defines the location on the longitudinal axis and between the entry portion and the end portion.

8. The apparatus of claim 1, wherein the end portion defines an end surface substantially facing the entry portion, and wherein the end surface is configured to elastically deform in response to a force imparted substantially parallel to the longitudinal axis.

9. The apparatus of claim 1, wherein the housing defines a guide surface extending from the entry portion, wherein the guide surface extends in a direction substantially parallel to or coincident with the longitudinal axis.

10. The apparatus of claim 1, wherein the fitting defines a fitting opening which opens into the fluid chamber, wherein the opening is configured to receive a device, and wherein the fluid chamber is configured to receive the fluid from the device when the fitting opening receives the device.

11. The apparatus of claim 10, wherein the fitting is configured to engage the device to form a fluid-tight seal with the device when the fitting opening receives the device.

12. The apparatus of claim 10, further comprising the device, wherein the device is configured to pressurize the fluid in the fluid chamber when the fluid chamber receives the fluid from the device, and wherein the fitting is configured to provide the fluid to the one or more fluid channels when the device pressurizes the fluid in the fluid chamber.

13. The apparatus of claim 12, wherein the device is a syringe.

14. The apparatus of claim 1, wherein the outer surface of the housing supports the fitting.

15. The apparatus of claim 1, wherein the fitting supports one or more fluid channel openings which open into the fluid chamber, wherein the one or more fluid channel openings are fluidly coupled to the one or more fluid channels.

16. An apparatus, comprising:
a housing, the housing defining an outer surface and an inner surface opposite the outer surface, the inner surface defining an inner chamber, and the housing defining a longitudinal axis extending through the inner chamber, wherein:
the housing defines an entry passage intersected by the longitudinal axis, the entry passage extending from the outer surface to the inner surface,
the housing supports a one or more fluid channels, each fluid channel defining a spray outlet which opens into the inner chamber, and each spray outlet configured to provide a spray of a fluid into the inner chamber when the one or more fluid channels receives the fluid,
the housing supports a magnification pane configured to provide visual access through the housing and into the inner chamber,
the housing supports the magnification pane to cause a field of view of the magnification pane to encompass a portion of the inner chamber through which the longitudinal axis extends, and
the magnification pane defines an optical axis substantially perpendicular to the longitudinal axis; and
a fitting defining a fluid chamber configured to hold the fluid, wherein the fluid chamber is configured to provide the fluid to the one or more fluid channels.

17. The apparatus of claim 16, wherein the fitting defines a fitting opening which opens into the fluid chamber, wherein the opening is configured to receive a device, and wherein the fluid chamber is configured to receive the fluid from the device when the fitting opening receives the device.

18. The apparatus of claim 16, wherein the magnification pane defines a visual marker, the visual marker configured to define a location within the field of view of the magnification pane and on the longitudinal axis when a vector substantially perpendicular to the longitudinal axis passes through the visual marker.

19. A method, comprising:
providing, using a fitting defining a fluid chamber, a fluid to a one or more fluid channels supported by a housing;
providing, using the one or more fluid channels, at least one spray of a fluid into an inner chamber defined by an inner surface of the housing, wherein the housing defines a longitudinal axis extending through the inner chamber, wherein the housing defines an entry passage opening into the inner chamber and intersected by the longitudinal axis, and wherein the entry passage extends between the inner surface and an outer surface defined by the housing and opposite the inner surface; and
providing, using a magnification pane supported by the housing, visual access through the housing and into the inner chamber.

20. The method of claim 19, further comprising causing, using the magnification pane, a field of view to encompass a portion of the inner chamber through which the longitudinal axis extends.
